(19) United States
(12) Patent Application Publication
(10) Pub. No.: US 2009/0326391 A1
(43) Pub. Date: Dec. 31, 2009

(54) WRIST-TYPE BLOOD PRESSURE METER

(75) Inventors: Chung Man Chan, Kowloon (HK); Ching Ching Cheung, Kowloon (HK); Ka Lun Fan, Kowloon (HK); Hung Keung Tse, Kwung Tong (HK)

Correspondence Address: DICKE, BILLIG & CZAJA, FIFTH STREET TOWERS, 100 SOUTH FIFTH STREET, SUITE 2250, MINNEAPOLIS, MN, 55402, US

(73) Assignee: Aviton Care Limited, Kwun Tong (HK)

(21) Appl. No.: 12/348,515

(22) Filed: Jan. 5, 2009

Related U.S. Application Data
(63) Continuation of application No. 61/018,903, filed on Jan. 4, 2008.

(51) Int. Cl. A61B 5/022

(52) U.S. Cl. 600/490

(57) ABSTRACT

A wrist-type blood pressure meter for measuring blood pressure and heart rate of a person and displaying the measurement reading with segmented screens showing numeric figures and graphical plot. It has memory module and fingerprint module allowing more than one user to store and retrieve measurement readings according to time sequence and calculate average readings.

### REFERENCE TO RELATED APPLICATION

This application claims priority to U.S. Provisional Applic. No. 61/018,903, filed Jan. 4, 2008, the contents of which are incorporated herein by reference.

### FIELD OF THE INVENTION

The invention relates generally to a blood pressure meter. More particularly, the invention relates to a wrist-type blood pressure meter with multi-screens graphical display.

### BACKGROUND OF THE INVENTION

In a conventional electronic blood pressure meter, there is display means displaying the measured blood pressure. However, the users often need to refer to the instruction booklet or have to familiarize themselves with the meaning of the blood pressure readings to understand the meaning thereof. The numbering of the blood pressure readings alone may not be good enough. Further, as blood pressure problem becoming more common among people, there is a greater likelihood that more than one user is using one same blood pressure meter in a family. Accordingly, it is vital to be able to allow more than one user to read the results of measurement over a number of tracked blood pressure readings.

Several U.S. patents and patent publications disclose blood pressure monitors for measuring and displaying the readings. Examples of these references include Terumo Kabushiki Kaisha, U.S. Pat. No. 4,747,412 and Omron Corporation, U.S. Pat. Nos. 5,687,732 and 6,547,741.

Each of these blood pressure monitors measures a user's blood pressure. The blood pressure monitors also generally include a display for displaying the blood pressure measurement readings.

### SUMMARY OF THE INVENTION

The wrist-type blood pressure meter with multi-screens graphical display is used to measure a user's systolic pressure, diastolic pressure and heart rate measurement. More than one user identifies herself by fingerprints and accordingly reads and stores her blood pressure measurement readings respectively. The wrist-type blood pressure meter has more than one LCD display visualizing the blood pressure measurement reading in easy-to-read figures and graphical plot. The wrist-type blood pressure meter also alerts the user by giving visual and audible warning when false measurement reading is detected.

### BRIEF DESCRIPTION OF THE DRAWINGS

FIG. 1 is a front view of a wrist-type blood pressure meter according to an embodiment of the invention that is adapted for comprising dual displays.

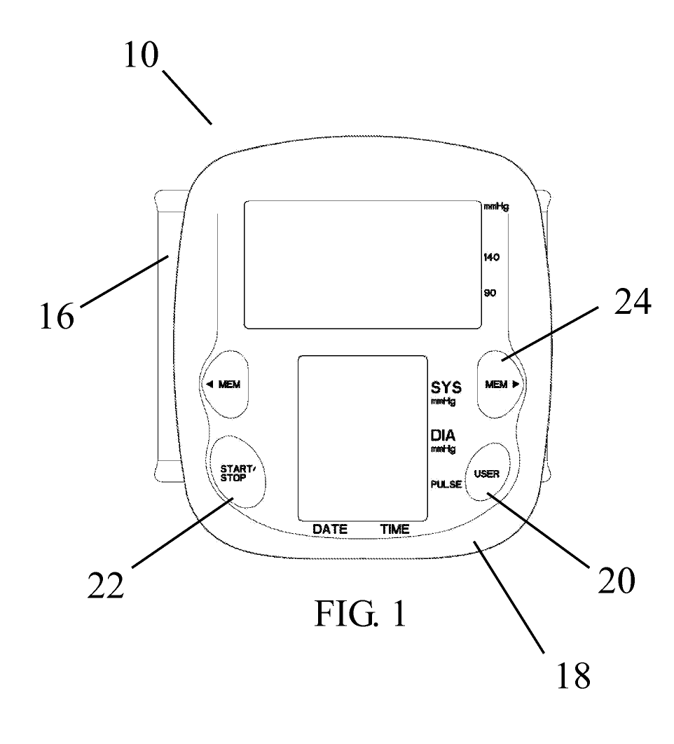

FIG. 2 is a side view of the wrist-type blood pressure meter of FIG. 1.

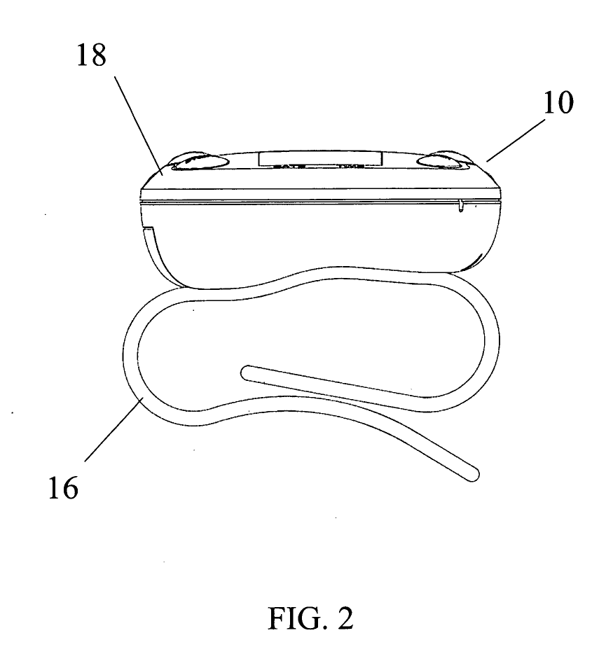

FIG. 3 is a block diagram of aspects of the wrist-type blood pressure meter.

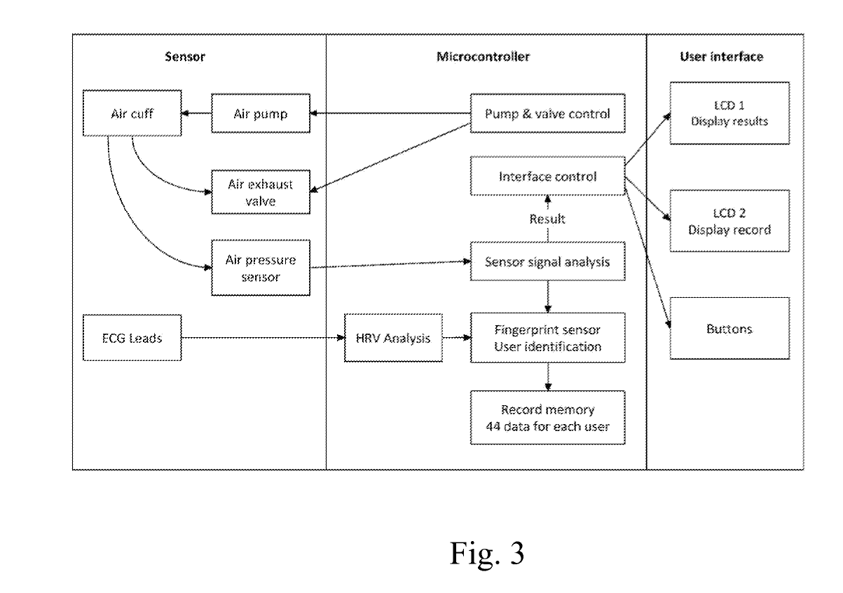

FIG. 4 is a flow diagram illustrating operation of the wrist-type blood pressure meter.

FIG. 5 is a LCD display of the wrist-type blood pressure meter showing numeric and symbolic figures;

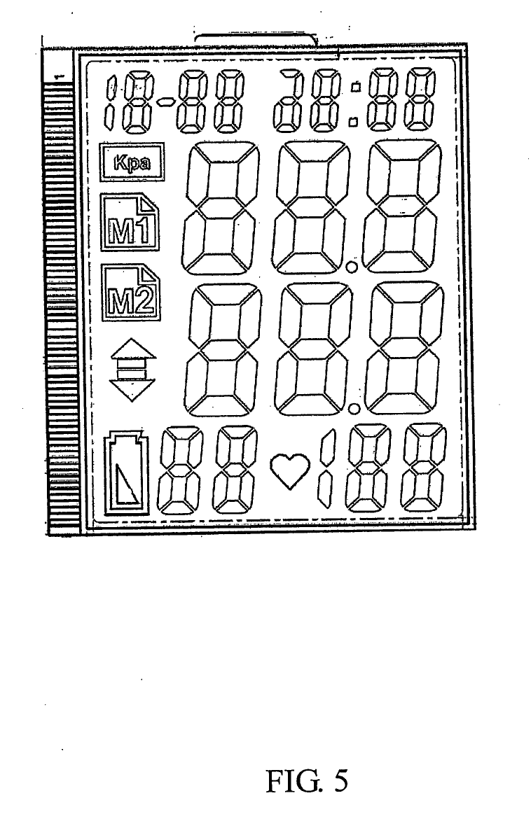

FIGS. 6a, 6b, 6c and 6d and are displays of the wrist-type blood pressure meter showing graphical output of blood pressure reading in a series of symbols showing systolic pressure, diastolic pressure and over limit pressure respectively.

FIGS. 7a, 7b, 7c, 7d, 7e, 7f and 7g illustrates various measurement readings of the LCD display of FIG. 5.

### DETAILED DESCRIPTION OF THE PREFERRED EMBODIMENTS

The invention is a wrist-type blood pressure meter 10, as illustrated at 10 in the Figures. The wrist-type blood pressure meter 10 thereby facilitates providing measurement of blood pressure, heart rate variability, date and time display and display of measurement reading records. It further detects and alerts the user of measurement error. The wrist-type blood pressure meter 10 accommodate more than one user and each user identifies herself by fingerprint.

The wrist-type blood pressure meter 10 generally includes an outer enclosure 18 in which the components of the wrist-type blood pressure meter 10 are contained. The outer enclosure 18 may be fabricated from a variety of materials depending on the desired appearance of the wrist-type blood pressure meter 10 and the conditions under which the wrist-type blood pressure meter 10 is to be used. In one configuration, the outer enclosure 18 is fabricated from injection molded plastic. In the preferred embodiment, the wrist-type blood pressure meter 10 further includes a cuff 16 which is firmly attached to the outer enclosure 18 and encircling the wrist of the user. Cuff 16 is adapted to encircle the wrist and to be adjustable to fit different circumferences of the wrists of each user with a hook-and-loop fastener (not shown) or alternatively with elastic materials of a circumference typically smaller than the circumference of wrists of different users.

A user button 20 may occupy a relatively small portion of at least one surface of the outer enclosure 18 to allow the user to depress the user button 20 for fingerprint recognition mechanism. The fingerprint recognition mechanism is known. The user button incorporating the fingerprint recognition mechanism allows a multiple of users to store, retrieve and read their own measurement readings record.

A start/stop button 22 may also be provided on the outer enclosure 18. The start/stop button 22 may be disposed at the other proximal edge of the user button 20.

Inside of the outer enclosure 18 components necessary for the operation of the wrist-type blood pressure meter 10 are located. The components may include a microprocessor, a blood pressure measuring device, a ECG leads device measuring heart rate variability, an air cuff sensor, a sound generation device, more than one LCD display with light emission display device, a date and timer device, a measurement error detection device, a fingerprint sensor, a calculation device and a memory device. In one embodiment, the LCD with light emission display device is a segmented dual display, wherein the first display shows the measurement data in numeric figures and symbols and the second display showing measurement reading in graphical plots.

In one embodiment, the sound generation device is a speaker. In one embodiment, the measurement error detection device is adapted to detect and send electronic signals to the microprocessor upon detection of failure to measure blood pressure or heart rate, air leakage in the air cuff, cuff loose, user body movement error and measurement reading above 260 mmHg. In one embodiment, the memory device is capable of storing and retrieving of measurement readings of a multiple of users. In one embodiment, the calculation device is adapted to calculate the average value of the last three measurement readings of a selected user and send such average value to the microprocessor and then the LCD display for displaying. To enhance the mobility of the wrist-type blood pressure meter 10, the components of the wrist-type blood pressure meter 10 may be powered by battery. The wrist-type blood pressure meter 10 may include the ability to recharge the battery. The LCD display is adapted to show battery warning 26 upon receiving electronic signal from the microprocessor of low power.

In one embodiment, the user may select blood pressure display unit in mmHg and kpa.

The wrist-type blood pressure meter 10 is adaptable to attachment to the user using a variety of configurations. An important criterion would be that the method of attaching the wrist-type blood pressure meter 10 to the user's wrist is that the attachment mechanism enables firm attachment, thus stable measurement, to the user's wrist, as loose cuffing could lead to false measurement by the wrist-type blood pressure meter 10.

One particular configurations include encircling on a user's wrist and firmly attaching to the user's wrist by a hook-and-loop fastener (not shown) or alternatively with elastic materials of a circumference typically smaller than the circumference of wrists of different users. In operation, the wrist-type blood pressure meter 10 is enabled by attaching the wrist-type blood pressure meter 10 to a user's wrist using one of the mechanisms set forth above. Further or alternatively, the hook-and-loop fastener or the attachment element, e.g. an arm cuff, is adapted to fit the user's arm such that the blood pressure meter 10 can be used to encircle the user's arm as an arm-type blood pressure meter.

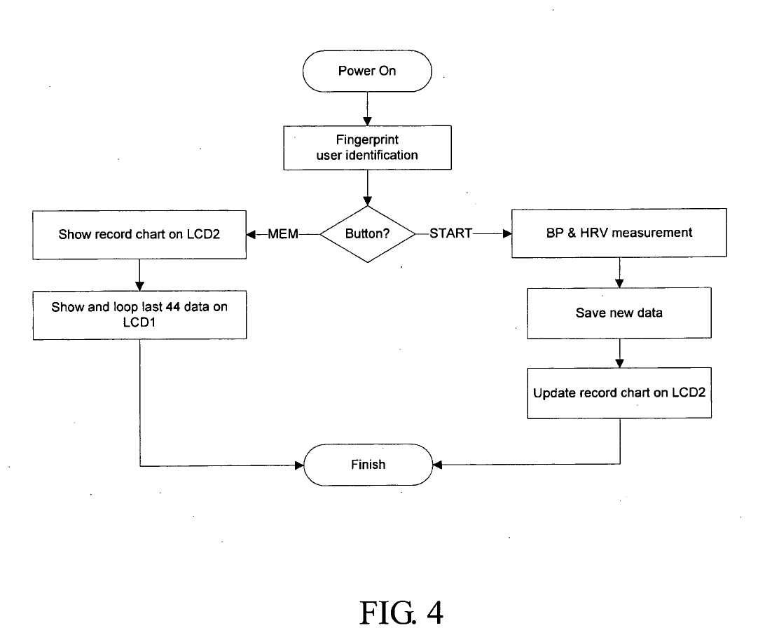

An operation flow diagram for the wrist-type blood pressure meter is illustrated in FIG. 4. The wrist-type blood pressure meter 10 may measure systolic pressure, diastolic pressure and/or heart rate variability of the user using the wrist-type blood pressure meter 10. For example, the wrist-type blood pressure meter 10 may be activated if the user presses the start/stop button 22 and presses the user button 20 identifying herself whereby the microprocessor selects the corresponding memory unit assigned to such user. In one embodiment, the user button is adapted to include a fingerprint sensor which recognizes the fingerprint of each user.

Alternatively or additionally, the wrist-type blood pressure meter 10 may also start measurement when presses the start/stop button 22. The measurement reading will be automatically shown and saved as the measurement reading of User 1.

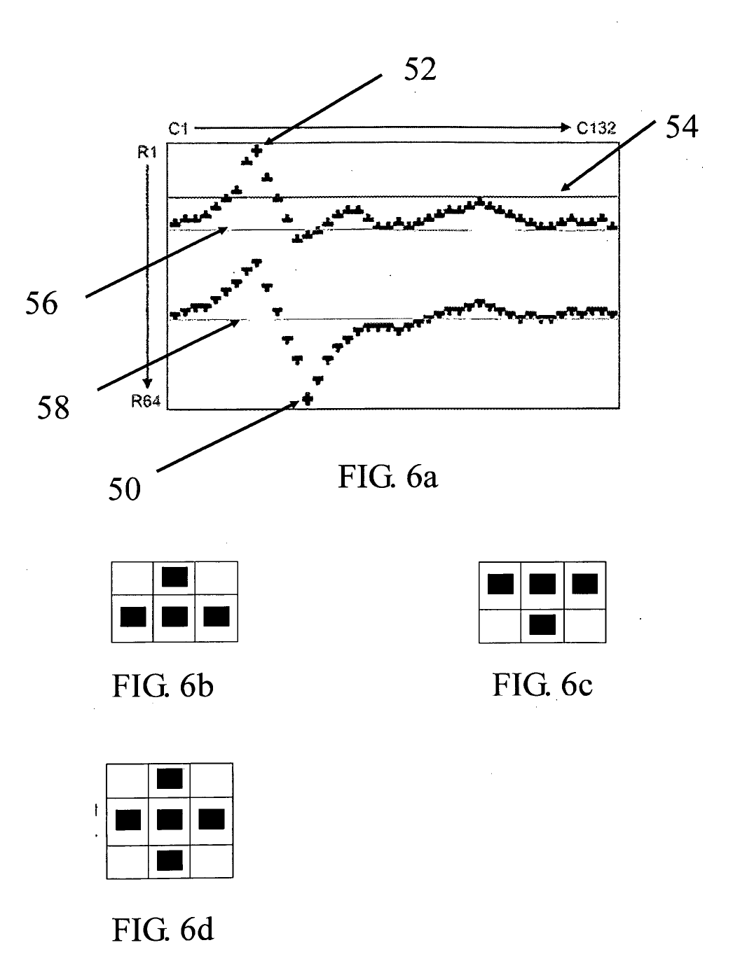

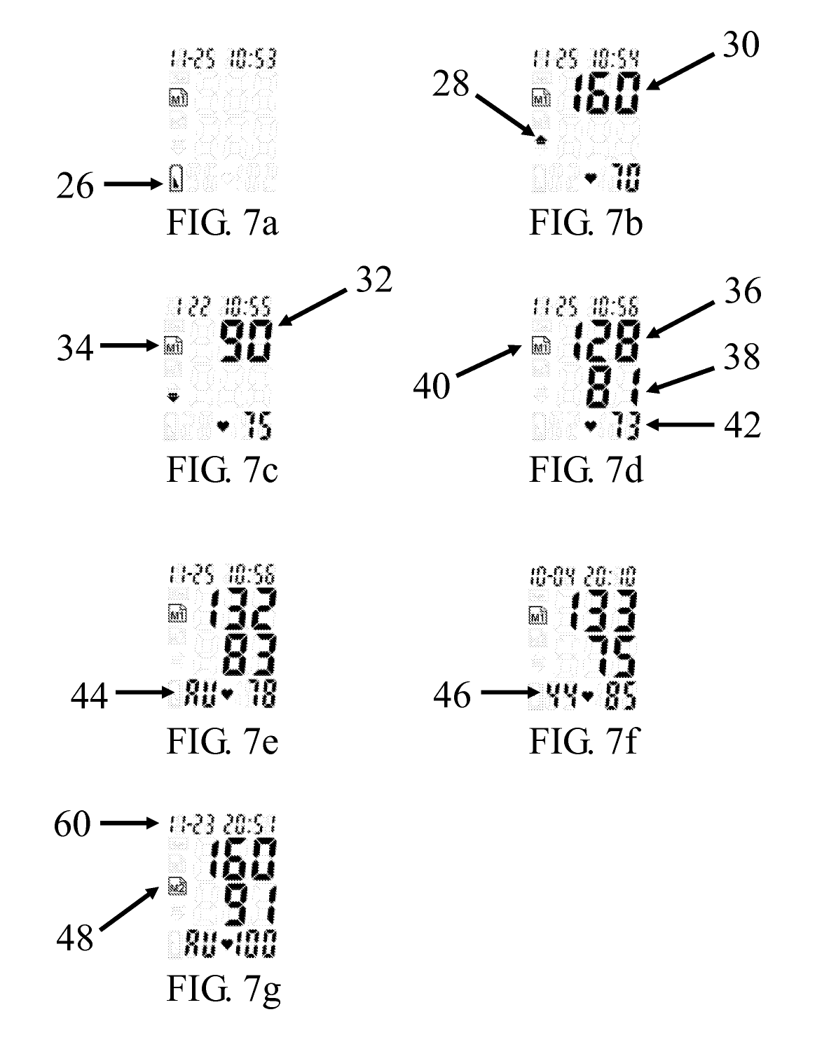

In operation, the wrist-type blood pressure meter 10 is enabled by attaching the wrist-type blood pressure meter 10 to a user's wrist using one of the mechanisms set forth above. When a user presses the start/stop button 22, the wrist-type blood pressure meter 10 automatically start measurement of a user's blood pressure and heart rate. In one configuration the measurement reading is automatically saved to User 1's memory unit. Alternatively, the user may press the user button 20 before or immediately after the measurement to interchangeably select the other user identity. Alternatively, the fingerprint sensor is disposed at the start/stop button 22 and/or the user button 20, the microprocessor receives electronic signal from the fingerprint sensor and selects the corresponding user identification and memory unit for saving and retrieval of the measurement reading of the selected user. The wrist-type blood pressure meter 10 then update the measurement reading records in the memory unit and display the measurement reading in numeric fashion (as shown in FIG. 7) and graphical plot pattern (as demonstrated in FIG. 6).

The sounds alarm, further and/or alternatively be accompanied by a light displayed on the wrist-type blood pressure meter 10, is activated upon detecting measurement errors, such as failure to obtain electronic signals of measurement reading or pressure reading over a pre-determined level, e.g. 260 mmHg. The sensor in air cuff is adapted to detect air leakage and cuff loose. Other error sensor may also be added, such as sensor to detect user body movement when measuring. The light can be generated from a variety of sources, utilizes a variety of colors and be displayed in a variety of patterns. One suitable light source is a red LED. One suitable display pattern is flashing to attract more attention to the light.

The wrist-type blood pressure meter 10 may be configured such that the measurement reading is timed and dated. The memory unit records the time and date of the measurement reading. The display unit shows the measurement reading with time and date 60. Further, a calculation unit is adapted to calculate an average value of the measurement readings of a pre-determined number of records. The user may retrieve the records of measurement reading and average value thereof by momentarily pressing the memory button 24.

FIG. 7a illustrates the LCD display in one embodiment where the low battery icon 26 is on indicating low battery. FIG. 7b illustrates an example when the measurement is conducting and the increasing measurement pressure is indicated by the level of 160 mmHg 30 and by an up arrow 28. FIG. 7c illustrates an example when the measurement is conducting and the decreasing measurement pressure is indicated by the level of 90 mmHg 32 and by a down arrow 34. FIG. 7d illustrates an example of numeric display of measurement reading of User 1 40 where systolic pressure is 128 mmHg 36 and the diastolic pressure is 81 mmHg 38 and the heart rate is 73 42. FIG. 7e illustrates an example of showing the average value of selected recorded heart rate at 78. FIG. 7f illustrates an example of showing a 44th old record 46 of measurement readings. FIG. 7g illustrates an example of showing the measurement reading of user 2 48.

FIG. 6a illustrates the second LCD display in one embodiment where the measurement reading of blood pressure is represented in graphical plot pattern having the pressure range of 50 mmHg at the lowest and 205 mmHg at the highest along its vertical axis and sequence of time of measurement whereby the oldest record on the right and the latest record on the left along its horizontal axis. In preferred embodiment, the vertical axis is divided into 64 units (R1-R64) and each unit (row pixel on display) represents 2.5 mmHg. 50 mmHg 50 therefore positions at the level of R63. 205 mmHg 52 positions at the level of R1. The horizontal axis is divided into 132 units (C1-C132) and the calculator device divides the number of available records by 132 and thereby distributing the corresponding measurement readings over the 132 units along the horizontal axis. In the example of 44 available recorded measurement readings, the latest measurement reading is therefore positioned in the region of C1-C3, the second latest measurement reading is positioned in the region of C4-C6 and the 44^{th} measurement reading is positioned in the region of C130-C132. FIG. 6b illustrates the graphical presentation of systolic pressure. FIG. 6c illustrates the graphical presentation of diastolic pressure. FIG. 6d illustrates the graphical presentation of the readings outside pre-determined ranges of pressure.

Alternatively or additionally, three pre-set lines are disposed on the transparent cover of the second display of graphical plot. One red line is at the level of 180 mmHg 54. Two yellow lines are at the respective levels of 140 mmHg 56 and 90 mmHg 58. Such lines are for indicating the normal and abnormal blood pressure level. Alternatively, the lines are adapted to be subject to user's change and flexibly disposed on the second display screen.

In the preferred embodiment, when the first display is displaying a measurement reading, the corresponding portion of graphical plots of the same measurement reading is adapted to be flashing or alternatively, in a different color. The user may easily find reference of her track and trend of a selected measurement reading.

It is contemplated that features disclosed in this application, as well as those described in the above applications incorporated by reference, can be mixed and matched to suit particular circumstances. Various other modifications and changes will be apparent to those of ordinary skill.

1. A wrist-type blood pressure meter comprising:
a microprocessor module;
a screen module, wherein the screen comprises at least two displays visualizing measurement readings of systolic pressure, diastolic pressure and pulse rate in numeric figures and graphical plot and the screen module is operably connected to the microprocessor module;
a user identity module for selecting the user identity and the corresponding historical measurement readings records, wherein the user identity module is operably connected to the microprocessor module;
an outer enclosure;
an air cuff attached to a lower wall of the outer enclosure;
an operation module, wherein the operation module is operably connected to the microprocessor module and comprises a start/stop button on one side of an upper wall of the outer enclosure and a memory button and an user selection button on the other side of an upper wall of the outer enclosure;
a blood pressure measuring module, wherein the blood pressure measuring module is operably connected to the microprocessor module;
a ECG leads module for measuring heart rate variability, wherein the ECG leads module is operably connected to the microprocessor module; and
a line indication module for indicating normal and abnormal blood pressure, wherein the indication lines are disposed on one display displaying graphical plot.

2. The wrist-type blood pressure meter of claim 1, and further comprising a memory module that is operably connected to the microprocessor module.

3. The wrist-type blood pressure meter of claim 1, and further comprising a timer module that is operably connected to the microprocessor module.

4. The wrist-type blood pressure meter of claim 1, and further comprising an alert module that is operably connected to the microprocessor module.

5. The wrist-type blood pressure meter of claim 1, and further comprising a battery module.

6. The wrist-type blood pressure meter of claim 4, wherein the alert module is capable of transmitting visual and audible warning when having detected error measurement.

7. The wrist-type blood pressure meter of claim 1, wherein the user identity module determines user identity by fingerprint, the fingerprint sensor being disposed on at least one button of the operation module.

8. The wrist-type blood pressure meter of claim 1, wherein the attaching means for attaching the air cuff is a hook-and-loop fastener.

9. The wrist-type blood pressure meter of claim 1, wherein the attaching means for attaching the air cuff is elastic materials of a circumference.

10. The wrist-type blood pressure meter of claim 1, and further comprising a sensor detecting air leakage and cuff loose.

11. A method of measuring blood pressure and heart rate of a person using a wrist-type blood pressure meter, wherein the method comprises:
encircling a wrist-type blood pressure meter to a person's wrist whose blood pressure and heart rate is to be measured;
pressing a start button on the wrist-type blood pressure meter, wherein the start button is operably connected to the microprocessor module;
measuring the blood pressure and heart rate of the person with a blood pressure measuring module and a ECG leads module, wherein the blood pressure measuring module and the ECG leads module is operably connected to the microprocessor module; and
displaying the measurement reading in numeric figures and graphical plot with a screen module comprising at least two displays, wherein the screen module is operably connected to the microprocessor module.

12. The method of claim 11, and further storing and retrieving data relating to historical measurement reading records in a memory module, wherein the memory module is operably connected to the microprocessor module.

13. The method of claim 11, and further comprising time and date of measurement.

14. The method of claim 11, wherein one display of the screen module has its vertical axis being divided into 64 units and each unit representing 2.5 mmHg and its horizontal axis being divided into 132 units and each three units indicating one measurement reading record, the oldest measurement reading record on the right and the latest record on the left thereby showing the time sequence of measurement reading records.

15. The method of claim 14, wherein the latest record is adapted to be flashing or in one color different from the color of older records which guide a user to the latest record.

16. The method of claim 11, wherein the screen module shows the measurement readings in the blood pressure display unit in kpa.

17. The method of claim 11, wherein the alert module upon detecting the measurement reading is above 260 mmHg initiates an alert comprises visual and audible warning indicating error measurement.

18. The method of claim 11, wherein the alert module upon detecting air leakage and cuff loose initiates an alert comprises visual and audible warning indicating air leakage and cuff loose.

19. The method of claim 11, wherein further displaying systolic values by upward symbol, diastolic values by downward symbol and over limit readings by a cross symbol.

20. The method of claim 11, wherein the screen module further comprises three pre-set lines, disposed on a transparent cover of one display displaying graphical plot, indicate the normal and abnormal blood pressure level; the first line in one color is at the level of 180 mmHg, indicating any measurement reading above the first line is in the area of abnormal high blood pressure; the second line and the third line in another color are at the level of 140 mmHg and 90 mmHg respectively, indicating any measurement reading between the two lines is in the area of normal blood pressure.

21. The method of claim 20, wherein the three lines are adapted to be subject to the adjustment.

22. The method of claim 11, and further recognizing a person's fingerprint with the fingerprint module, wherein the fingerprint module is operably connected to the microprocessor module and is capable of selecting at least two users' identities and the corresponding memory units.

23. The method of claim 11, and further comprising
timing the measurements;
recording the measurement time and date;
storing the measurement readings; and
calculating the average value of the measurement readings.

24. The method of claim 23, and further comprising retrieving and displaying the historical measurement reading records with time and date and the average value of the measurement readings.
